(19) United States
(12) Patent Application Publication
(10) Pub. No.: US 2019/0351090 A1
(43) Pub. Date: Nov. 21, 2019

(54) SYSTEM AND METHOD FOR STERILIZING MEDICAL WASTE

(71) Applicants: Jean Louis GUIRETTE, Houston, TX (US); Robert GUIRETTE, Houston, TX (US); GALlA TEXTIL, Tlaxcala (MX)

(72) Inventors: Jean Louis GUIRETTE, Houston, TX (US); Robert GUIRETTE, Houston, TX (US)

(73) Assignees: GALIA TEXTIL, Tlaxcala (MX); Jean Louis GUIRETTE, Tlaxcala (MX)

(21) Appl. No.: 16/475,812

(22) PCT Filed: Jan. 4, 2018
(86) PCT No.: PCT/US2018/012275

§ 371 Date: Jul. 3, 2019

Related U.S. Application Data
(60) Provisional application No. 62/443,446, filed on Jan. 6, 2017.

(51) Int. Cl. A61L 11/00; B09B 3/00; A61L 2/07; A61L 2/26

(52) U.S. Cl. CPC A61L 11/00; B09B 3/0091; A61L 2/07; A61L 2/26; A61L 2202/11; A61L 2202/123; A61L 2202/14; A61L 2202/16; A61L 2202/121

(57) ABSTRACT

A system for sterilizing medical waste includes a pressure tank configured to receive a compression bag having the medical waste; a water vapor generator that introduces steam into the pressure tank via a first pipe using a connector attached to the pressure tank; and a vacuum compressor that removes fluids from the pressure tank. The pressure tank, water vapor generator, and vacuum compressor are connected in a closed manner such that the fluids within the pressure tank are contained.

### RESERVATION OF COPYRIGHTS

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

Public and private health institutions, including clinics, hospitals, research facilities, etc., produce a large amount of medical waste as a result their daily activities. According to industry practice, the medical waste can be categorized as anatomical waste (e.g., body parts and organs) or non-anatomical waste (e.g., sharps that have been in contact with animal or human blood, biological fluids, tissues, cultures, live vaccines, containers or materials saturated with blood products). According still to industry practice, the medical waste can also be categorized as either risk waste or non-risk waste. The risk waste is further divided into 7 groups: (1) infectious waste, (2) pathological waste, (3) sharps, (4) pharmaceutical waste, (5) genotoxic waste, (6) chemical waste, and (7) radioactive waste.

Infectious waste is any waste that is contaminated by any type of bacterium, virus, parasites, or fungi. Examples of infectious waste include cultures, waste from surgery and autopsies, waste from infected patients, waste from infected hemodialysis patients, infected animals from laboratories, and any material having been in contact with infected patients.

Pathological waste includes, for example, tissues, organs, body parts, fetuses, blood and body fluids, etc.

Sharps includes, for example, needles, syringes, scalpels, infusion sets, saws and knives, surgical blades, broken glass, any other items that can cut and puncture.

### SUMMARY

Medical waste can fall under one or more categories. Although the specification describes treatment of certain waste in a particular manner, one of ordinary skill in the art would recognize that, based on the disclosure of the present specification, the inventor's system and method may be applied to any and all medical waste.

According to one aspect of the invention, one or more embodiments disclosed herein relate to a system for sterilizing medical waste comprising a pressure tank configured to receive a compression bag having the medical waste; a water vapor generator that introduces steam into the pressure tank using a connector attached to the pressure tank; and a vacuum compressor that removes fluids from the pressure tank, wherein the pressure tank, water vapor generator, and vacuum compressor are connected in a closed manner such that the fluids within the pressure tank are contained.

In another aspect, one or more embodiments disclosed herein relate to a method for sterilizing medical waste comprising: placing a compression bag having medical waste inside a pressure tank; connecting the compression bag to a connector within the pressure tank; extracting fluids from the compression bag to create a vacuum-like environment therein; introducing steam into the compression bag; removing the steam from the pressure tank; storing the fluids extracted from the compression bag and the steam removed from the pressure tank; and cooling the compression bag within the pressure tank.

In another aspect, one or more embodiments disclosed herein relate to a sterilization system comprising: a pressure tank for containing medical waste to be sterilized; a waste water collector and waste air collector connected to the pressure tank; and a heated steam injection unit that injects heated steam into the pressure tank in one or more cycles so as to sterilize medical waste contained within the pressure tank, wherein, after each cycle, the water and air contained within the pressure tank is collected in the waste water collector and the waste air collector.

### BRIEF DESCRIPTION OF DRAWINGS

FIG. 1 shows a compression bag.

FIG. 2 shows a compression bag.

FIG. 3 shows a hospital red bag to be disposed in a compression bag.

FIG. 4 shows a compression bag placed inside a container and in a position to be filled.

FIG. 5 shows a sealed compression bag holding a hospital red bag.

FIG. 6A shows a mechanism for sealing a compression bag.

FIG. 6B shows a mechanism for sealing a compression bag.

FIG. 6C shows a cable tie for sealing a compression bag in an open position.

FIG. 6D shows a cable tie for sealing a compression bag in an open position.

FIG. 6E shows a cable tie for sealing a compression bag in a closed position.

FIG. 7 shows a storage that contains a plurality of sealed compression bags awaiting processing.

FIG. 8 shows a metallic container that serves as a protection mechanism to prevent the compression bag from contacting components of the sterilization autoclave.

FIG. 9 shows a metallic container having a protective case.

FIG. 10 shows a sealed compression bag being placed inside a metallic container.

FIG. 11 shows a sealed compression bag being placed inside a metallic container.

FIGS. 12A and 12B each show a metallic container disposed in a sterilization autoclave chamber.

FIG. 13 a unidirectional flow device that may be used in conjunction with a sealed compression bag.

FIG. 14 shows a unidirectional flow device inserted into a sealed compression bag.

FIG. 15 shows a unidirectional flow device inserted into a sealed compression bag.

FIG. 16A shows a metallic container.

FIG. 16B shows a top-down view of a through-hole of a cap of a metallic container.

FIG. 16C shows a compression bag placed inside a metallic container.

FIG. 16D shows a unidirectional flow device being introduced into the compression bag via an opening of the bag.

FIG. 16E shows force being applied to a disk of a unidirectional flow device such that a passage between an interior and an exterior of a metallic container is open.

FIG. 16F shows the absence of force being applied to a disk of a unidirectional flow device or when the pressure inside a sealed compression bag is greater than that outside a metallic container.

FIG. 16G shows the entire structure of FIG. 16E being placed inside a sterilization autoclave chamber.

FIG. 16H shows a cap of a metallic container.

FIG. 16I shows a unidirectional flow device.

FIG. 16J shows a unidirectional flow device working in conjunction with a compression bag.

FIG. 16K shows a connector that may be affixed to the pressure tank.

FIG. 16L shows a tube that can be used to introduce steam from the water vapor generator into the compression bag.

FIG. 17A shows a sealed compression bag holding a hospital red bag that is wrapped in a net, an upper portion of the net and the sealed compression bag being secured to a connector.

FIG. 17B shows the entire structure of FIG. 17A being affixed to a pressure tank via the connector.

FIG. 17C shows the structure of FIG. 17B with steam being injected via the connector, causing the sealed compression bag to become inflated.

FIG. 18 shows protective padding that is configured to receive sharps.

FIG. 19 shows an inner holder.

FIG. 20A shows an outer holder that is configured to hold the inner holder shown in FIG. 19.

FIG. 20B shows a group of circular through-holes aligned over a diametrical axis of a cap.

FIG. 20C shows a cap having non-circular through-holes.

FIG. 21 shows a protective padding being disposed at a bottom of an inner holder within the outer holder.

FIG. 22 shows a sharp, pointed object being inserted into the structure of

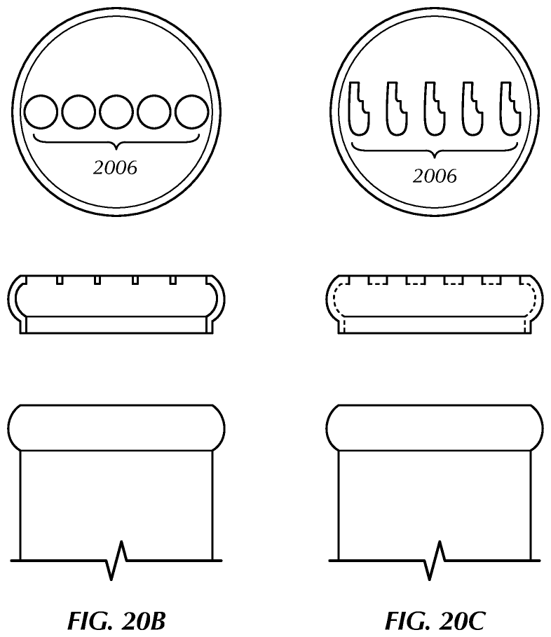

FIG. 20 via one of the structure's through-holes.

FIG. 23 shows sharps inside a cartridge.

FIG. 24 shows a capsule holding a cartridge.

FIG. 25 shows an encapsulation mechanism.

FIG. 26 shows a process of removing a cartridge from a capsule and placing the cartridge into a sterilization autoclave chamber.

FIG. 27A shows a deformed inner holder after having been subjected to sterilization treatment within a cartridge.

FIG. 27B shows a deformed inner holder after having been subjected to sterilization treatment.

FIG. 28 is a block diagram of a system for sterilizing medical waste.

FIG. 29 is a flow chart detailing a method of sterilizing medical waste.

FIG. 30A shows a system for sterilizing medical waste.

FIG. 30B shows a system for sterilizing medical waste.

### DETAILED DESCRIPTION

In the following detailed description, numerous specific details are set forth in order to provide a more thorough understanding of one or more embodiments of the invention. However, it will be apparent to one of ordinary skill in the art that the invention may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create a particular ordering of the elements nor to limit any element to being only a single element unless expressly disclosed, such as by the use of the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural referents unless the context clearly dictates otherwise. Thus, for example, reference to “a syringe” includes reference to one or more of such syringes. Further, it is to be understood that “or,” as used throughout this application, is an inclusive or, unless the context clearly dictates otherwise.

Terms like “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Inventors disclose a novel system and method for treating (i.e., sterilizing) infectious medical waste, non-anatomical medical waste, and sharps.

Specific embodiments will now be described in detail with reference to the accompanying figures. Like elements in the various figures are denoted by like reference numerals for consistency. Like elements may not be labeled in all figures for the sake of simplicity.

### Infectious Medical Waste

One or more embodiments of the invention relate to a system and method for treating infectious medical waste. Infectious medical waste may be treated using physical process, thermal process, chemical process, or a combination thereof to eliminate infectious characteristics and make such waste unrecognizable in shape and form. The system and method according to one or more embodiments of the invention are directed to creating a microclimate in a compression bag, thereby sterilizing all waste, whether liquid, solid, or gas, within the compression bag. According to one or more embodiments of the invention, the microclimate is created inside a hermetically sealed compression bag. Specifically, once the medical waste is hermetically sealed inside a compression bag, a sterilizing agent is introduced into the compression bag to facilitate sterilization of the waste. The system and method advantageously treat medical waste more efficiently and economically compared to existing systems and methods and comply with government regulations and laws governing the disposal of such medical waste around the world.

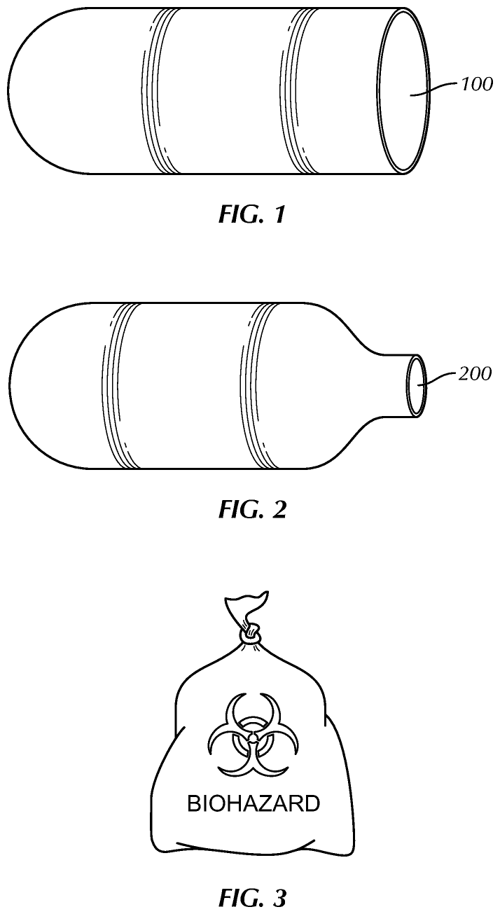

FIG. 1 shows a compression bag that is configured to hold hospital red bags. In general, a hospital red bag is used for the disposal of non-sharp and infectious medical waste. The compression bag has an opening (100). The compression bag may be made of elastic or elastomeric material including, but not limited to, latex, natural rubber, nitrile, polybutadiene, and polyurethane. Advantageously, once hermetically sealed, the bag allows for expansion of its walls due to internal forces generated by water vapor (during the sterilization process). Once internal forces are expelled, the bag's elastic walls return to their initial form and size while retaining their elasticity. Accordingly, because of the excellent mechanical properties of the compression bag, the walls of the compression bag do not break during sterilization processes that involve high temperature, thereby preventing waste from escaping from the interior of the compression bag and contaminating the surrounding environment. The compression bag is able to operate in temperature ranges from 100 to 150 degrees Celsius. The compression bag is also impermeable, thereby preventing wastes from permeating from the interior and contaminating the surrounding environment. One of ordinary skill in the art would appreciate that the dimension, color, and shape of the compression bag can vary depending on the specific needs of the health institution.

FIG. 2 shows a compression bag that is configured to hold hospital red bags. The compression bag of FIG. 2 is similar to FIG. 1. One difference is that the opening of the compression bag (200) of FIG. 2 is substantially smaller than the diameter of the compression bag. FIG. 1, on the other hand, shows a compression bag whose opening (100) is substantially equal to the diameter of the compression bag. The compression bag of FIG. 2 may allow for an easier closing of the compression bag.

FIG. 3 shows a hospital red bag to be disposed in the compression bag of FIG. 1 or FIG. 2. The hospital red bag is used for identification, separation, and packing of infectious medical waste. The hospital red bag is configured to handle non-anatomical waste. Packing of infectious medical waste is to be carried out in waterproof translucent red polyethylene bags each weighing a minimum of 200 grams. The bags are each marked with the universal biohazard symbol, with the legend “Infectious Biological Hazardous Waste,” and comply with the minimum tension resistance standards, elasticity, and torn resistance standards. In addition, such bags are configured to be filled up to 80% of their capacity and be closed before being transported to a temporary storage. The bag shall not be opened or emptied. As shown in FIG. 3, the hospital red bag is sealed and contains infectious medical waste.

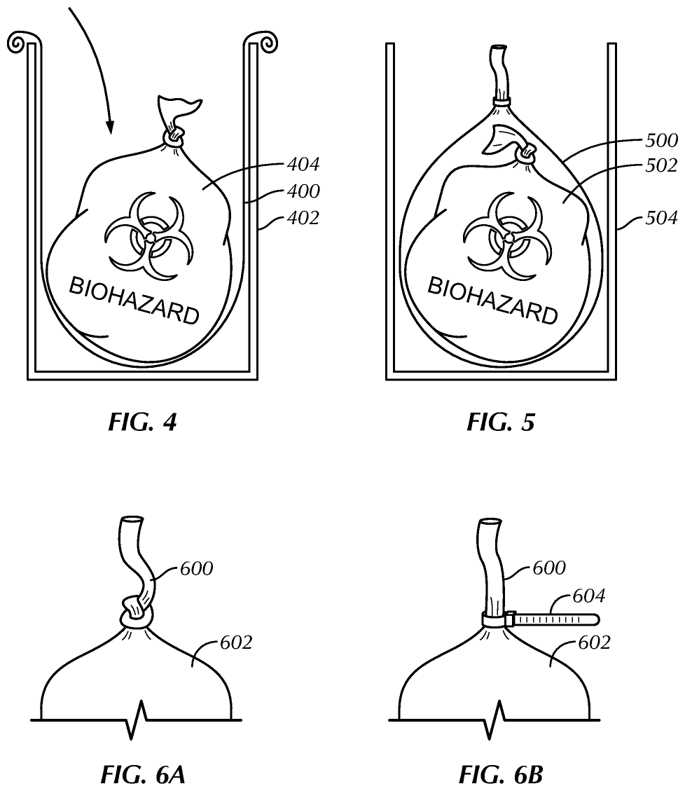

FIG. 4 shows a compression bag (400) placed inside a container (402) and in a position to be filled. Due to the elastic nature of the compression bag (400), a single person can easily place or remove the compression bag (400) from the container (402). FIG. 4 also shows a hospital red bag (404) being disposed in the compression bag (400). The interior wall of the container (402) (i.e., the wall in contact with the compression bag) may be coated with resin or rubber to smoothen the contact. Specifically, the interior of the container (402) may be treated such that the compression bag (400) is protected from tear, pierce, puncture, cut, break, or damage when being filled with hospital red bags (404). Although FIG. 4 only shows the container (402) holding one hospital red bag (404), the number is not limited and can vary depending on the size and shape of the container (402) and the compression bag (400).

FIG. 5 shows a sealed compression bag (500) holding a hospital red bag (502). The sealed compression bag sits inside the container (504).

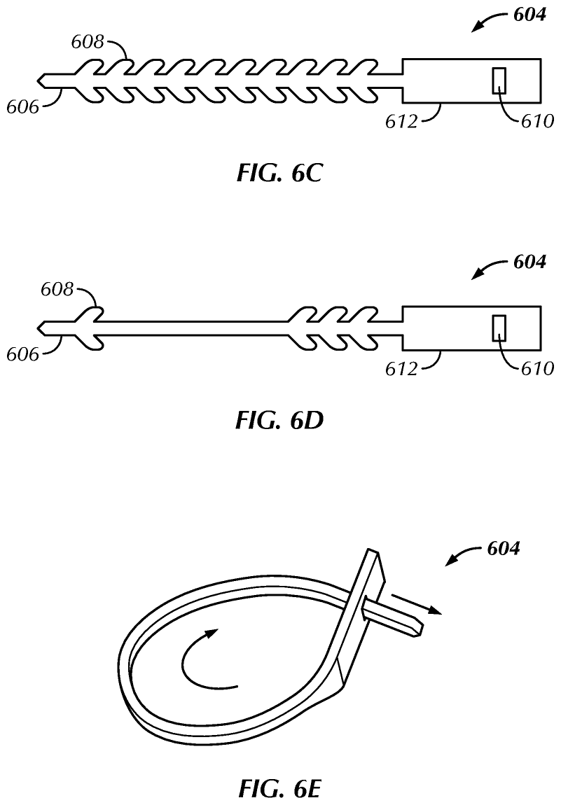

FIGS. 6A and 6B show two mechanisms for sealing the compression bag. One of ordinary skill in the art would appreciate that the compression bag may be hermetically sealed in any manner, not limited to those disclosed.

In FIG. 6A, the opening (600) of the compression bag (602) is closed by a simple knot. In FIG. 6B, the compression bag (602) is sealed by a cable tie (604), which may be made of plastic, elastomeric material of latex, vinyl, nitrile, polyurethane, or other materials that are elastic. The sealing mechanism is to hermetically seal the compression bag (602) so as to prevent medical waste from leaving the compression bag (602) as well as to provide sufficient flexibility to introduce an object (e.g., water, catalyst, etc.) into the compression bag (602).

FIG. 6C shows a cable tie (604) having 10 different locking positions (608). The cable tie (604) is used to seal the compression bag so that medical waste does not escape. A head part (606) is used to wrap around an opening of the compression bag. To seal the opening of the compression bag, the head part (606) is introduced into a hole (610) of the end part (612) and tightened using the various locking positions (608) based on the size of the opening of the compression bag.

FIG. 6D shows a cable tie (604) having 4 different locking positions (608). One difference from FIG. 6C is that, in FIG. 6D, the distance between successive locking positions may vary.

FIG. 6E shows the cable tie (604) of FIG. 6C or 6D in a closed position.

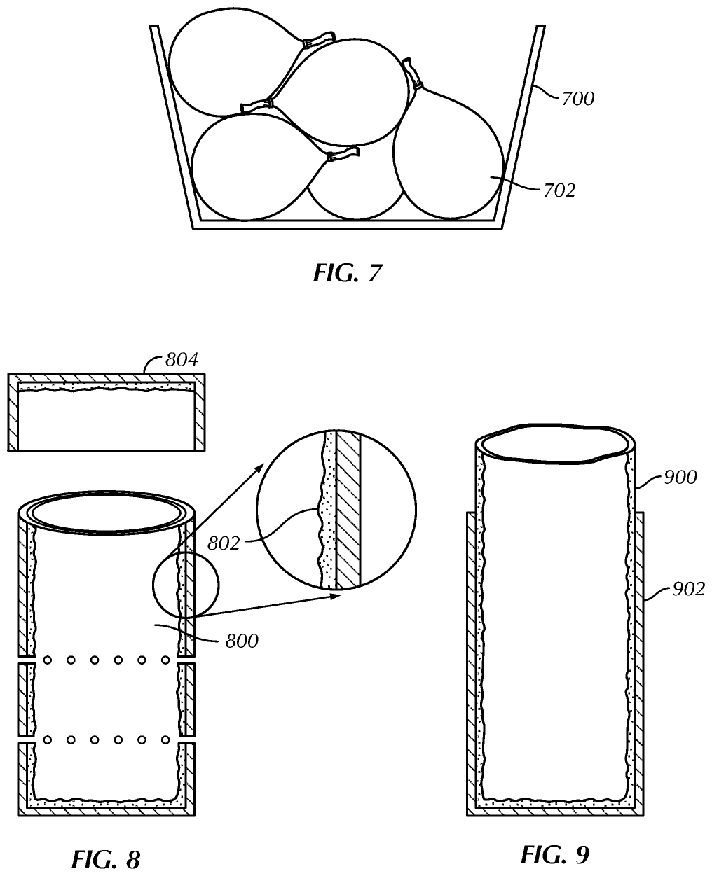

FIG. 7 shows a storage (700) that contains a plurality of sealed compression bags (702) awaiting processing. As with the interior wall of the container, the interior wall of the storage (700) (i.e., the wall in contact with the compression bags) may be coated with resin or rubber to smoothen the contact between the storage (700) and the sealed compression bags (702) such that the sealed compression bags (702) are protected from tear, pierce, puncture, cut, break, or damage.

When the time comes to sterilize the sealed compression bags (702) in a sterilization autoclave, the sealed compression bags (702) may first be placed into a metallic container. FIG. 8 shows a metallic container (800) that serves as a protection mechanism to prevent the compression bag from contacting components of the sterilization autoclave, including temperature sensors that could perforate the compression bag and electrical wires or power source that may scratch, damage, or even burn the compression bag.

As also shown in FIG. 8, the metallic container (800) is coated with protective coating (802) covering its entire interior surface, including the cap portion (804) on top. Such protective coating (802) eliminates rough edges, sharp ends, burrs, and any other roughness that may pierce, tear, cut, break, or damage the compression bag. Furthermore, the protective coating (802) prevents the sealed compression bag from making any contact with the sterilization autoclave. Various laboratory tests (including temperature tests that mimic the sterilization environment) illustrate damage and breakage to the sealed compression bag when the sealed compression bag directly contacts the metal portions of the sterilization autoclave or the metal portions of the metallic container (800). The protective coating (802) may be vulcanized natural rubber, polyurethane, synthetic rubber, or any other known material having similar properties for providing protection.

FIG. 9 shows a metallic container (900) having a protective case (902). The protective case (902) is used to isolate its contents from any rough edges, sharp ends, burrs, and any other roughness. As with the protective coating, the protective case (902) in FIG. 9 prevents the compression bag from contacting components of the sterilization autoclave, including temperature sensors that could perforate the compression bag and electrical wires or power source that may scratch, damage, or even burn the compression bag. Furthermore, the protective case (902) prevents the sealed compression bag from making any contact with the sterilization autoclave. Various laboratory tests (including temperature tests that mimic the sterilization environment) illustrate damage and breakage to the sealed compression bag when the sealed compression bag directly contacts the metal portions of the sterilization autoclave. The protective case (902) may be made from cotton, rayon, cotton-rayon polyester mix woven fabric, non-woven polypropylene fabric, cellulosic fibers fabric, or any other known material having similar properties for providing protection.

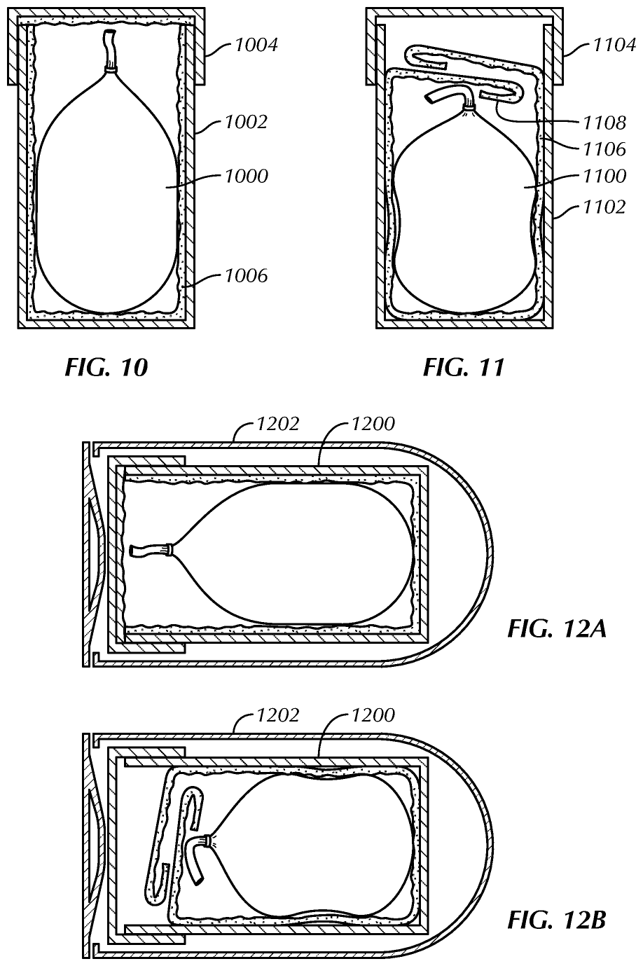

FIG. 10 shows a sealed compression bag (1000) being placed inside a metallic container (1002). The metallic container is closed with a cap (1004). The metallic container (1002) is coated with protective coating (1006). The entire structure is now ready to be placed into a sterilization autoclave for treating.

FIG. 11 shows a sealed compression bag (1100) being placed inside a metallic container (1102). The metallic container (1102) is closed with a cap (1104). Also shown in FIG. 11 is the sealed compression bag (1100) being enclosed inside a closed protective case (1106). The entire structure is now ready to be placed into a sterilization autoclave for treating. Note that the dimension of the protective case (1106) is not limited and can exceed that of the metallic container (1102) such that there are folds (1108) in the opening of the protective case (1106). Note that, different from FIG. 10, the metallic container (1102) in FIG. 11 does not have protective coating on its interior wall. However, one of ordinary skill in the art would appreciate that the protective coating and the protective case may be used in combination.

FIGS. 12A and 12B each show a metallic container (1200) disposed in a sterilization autoclave (1202). FIG. 12A shows the structure of FIG. 10 placed in a sterilization autoclave (1202). FIG. 12B shows the structure of FIG. 11 placed in a sterilization autoclave (1202).

One or more embodiments of the invention relate to placing medical waste in a sealed environment and sterilizing the content at high temperature to neutralize any infectious characteristics of the medical waste. The sterilization process is carried out in a sterilization autoclave chamber that is configured to withstand pressure created by water vapor at temperatures ranging from 100 to 200 degrees Celsius. In general, sterilization temperatures by water vapor are between 121 and 134 degrees Celsius. To achieve sterility, medical waste is heated in a chamber by injected steam until the waste reaches a time and temperature setpoint. The medical waste is then maintained at the setpoint for a period of time depending on the bioburden present and its resistance to steam sterilization. Sterilization is aimed at reducing the amount of microorganism or other potential pathogens that may be present in the waste. The degree of sterilization may be expressed by multiples of the decimal reduction time, or D-value, denoting the time needed to reduce the initial number Nₒ of microorganism and pathogen to one tenth of its original amount. The sterility assurance level (i.e., the maximum allowable amount of microorganism and pathogen present that qualifies the treated waste as non-infectious) for each jurisdiction and waste category can vary. Upon cooling, the sterilized compression bag can be removed for disposal or further processing.

Dimension of the sterilization chamber can vary depending on the need of the health institution. The sterilization capacity of the sterilization autoclave chamber (i.e., the amount of sterilizing agent the sterilization system holds) may be at least 5 liters, for example, and can vary depending on the need of the health institution. According to one or more embodiments of the present invention, the sterilization capacity of the sterilization autoclave chamber may be between 20 liters and 40 liters. The sterilizing agent may be stored in a container separate from the sterilization autoclave chamber or may be a portion of the sterilization autoclave chamber. The container storing the sterilizing agent may be a vessel, a reservoir, etc., and can vary depending on the nature of the agent (e.g., chemical additive, water, etc.). In one or more embodiments the agent is water condensed from water vapor used in a previous sterilization cycle. Accordingly, there may be a pipe, device, or mechanism that captures the condensation and reintroduces the same into a new batch of medical waste to be treated.

The sterilization autoclave chamber functions may require manual control or may be automated. The sterilization autoclave chamber functions may allow for customization. Specifically, the autoclave chamber may enable a user to select a temperature range of between 100 and 150 degrees Celsius for sterilizing infectious medical waste or enable a user to select a temperature range of between 100 and 200 degrees Celsius for sterilizing sharps. Furthermore, in addition to adjusting temperature, a user may set the amount of time for sterilizing medical waste, adjust the amount of water to be dispensed and introduced into the sealed compression bag, select the types, and the amount of chemical additives to be added, etc. One of ordinary skill in the art would recognize that the parameters disclosed above are merely illustrative purposes and can vary depending on the nature of the medical waste being treated, the thoroughness of the treatment, the needs of the health institution, etc.

Fluids, like water or chemical additive, may be introduced before or after sealing the compression bag. The compression bag may be filled by directly adding fluid into the opening or, if the compression bag has already been sealed, may be introduced using a unidirectional flow device. The unidirectional flow device enables water, water vapor, chemical additive, and other intended fluids to enter the compression bag, but prevents any medical waste that is already in the sealed compression bag to escape therefrom.

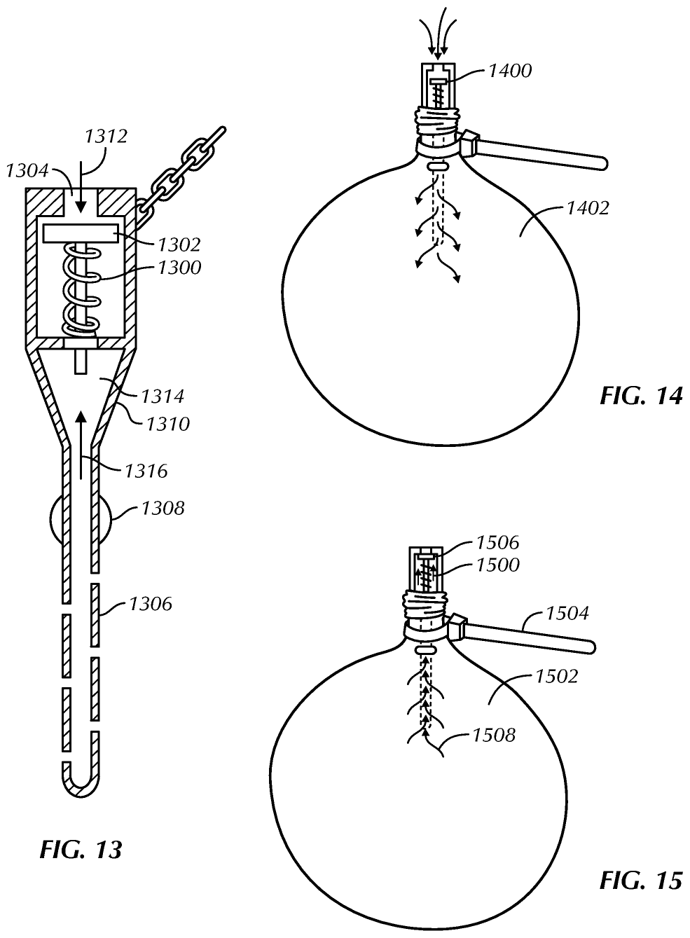

FIG. 13 shows an example of a unidirectional flow device that may be used in conjunction with a sealed compression bag. The unidirectional flow device comprises a spring (1300), a disk (1302), an opening (1304), a bottom portion (1306), a protrusion (1308), and a ramp (1310). The unidirectional flow device comprises an actuated closing mechanism in which the spring (1300) pushes against the disk (1302) to seal the opening (1304). To enable fluid to flow through the unidirectional flow device in a first direction (1312), force must act upon the disk (1302) such that there is a passage between the opening (1304) and the through portion (1314) of the unidirectional flow device. The actuated closing mechanism advantageously prevents medical waste from flowing in a second direction (1316). The bottom portion (1306) of the unidirectional flow device is configured to be inserted into the sealed compression bag. The external surface of the unidirectional flow device is smooth and free of rough edges, sharp ends, burrs, and any other surface roughness that may damage the compression bag's surface. The bottom portion (1306) may comprise a finishing known in the industry as “mirror finishing,” which allows for a smoother and easier introduction of the unidirectional flow device into the sealed compression bag. The protrusion (1308) is configured to pass through the cable tie and secure the unidirectional flow device against the sealed compression bag. Properly placed, the protrusion (1308) should be adjacent to the cable tie. The protrusion (1308) may be coated with adhesive. The ramp (1310) serves as a guide that prevents the user from inserting the unidirectional flow device too deeply into the sealed compression bag.

FIG. 14 shows a unidirectional flow device (1400) inserted into a sealed compression bag (1402). The sealed compression bag is sealed by a cable tie whose characteristics have been described. FIG. 14 also shows that the disk of the unidirectional flow device has been acted on and, accordingly, fluid can be added into the sealed compression bag in a first direction.

FIG. 15 shows a unidirectional flow device (1500) inserted into a sealed compression bag (1502). The sealed compression bag (1502) is sealed by a cable tie (1504) whose characteristics have been described. FIG. 15 also shows that the disk (1506) of the unidirectional flow device prevents any medical waste from attempting to flow in a second direction (1508). Thus, the medical waste cannot escape the sealed compression bag.

Although FIGS. 13-15 show a specific type of unidirectional flow device being used to introduce fluid into a sealed compression bag, other types of devices can be used. For example, a valve can be affixed to the opening of the sealed compression bag. For example, rather than using a unidirectional flow device, a bi-directional flow device may be used. For example, a tube can be affixed to the opening of the sealed compression bag and used to transport fluid into the sealed compression bag.

Once fluids have been introduced into the compression bag, the compression bag is ready to be treated. The sealed compression bag is placed into a metallic container, which is, in turn, placed into a sterilization autoclave shown in FIGS. 12A and 12B.

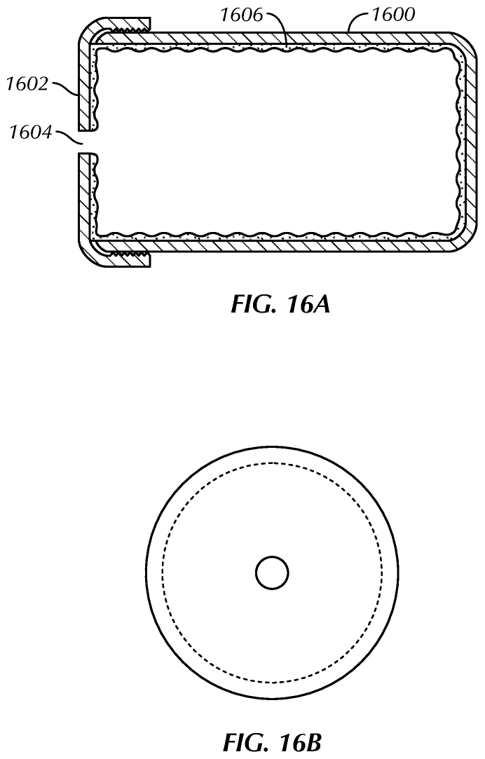

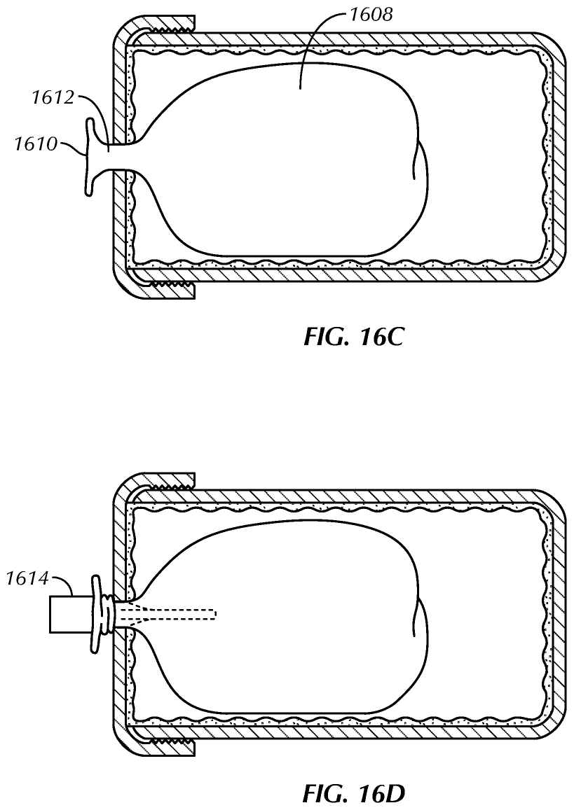

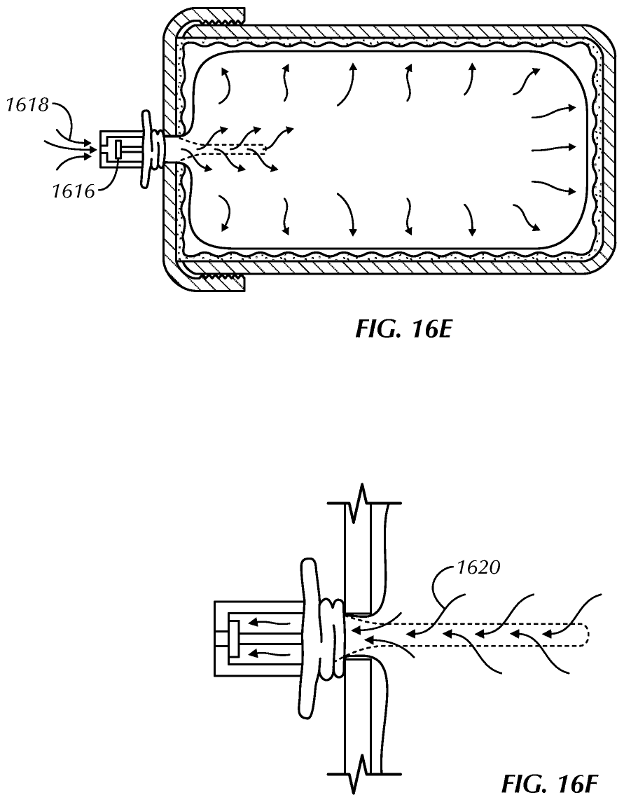

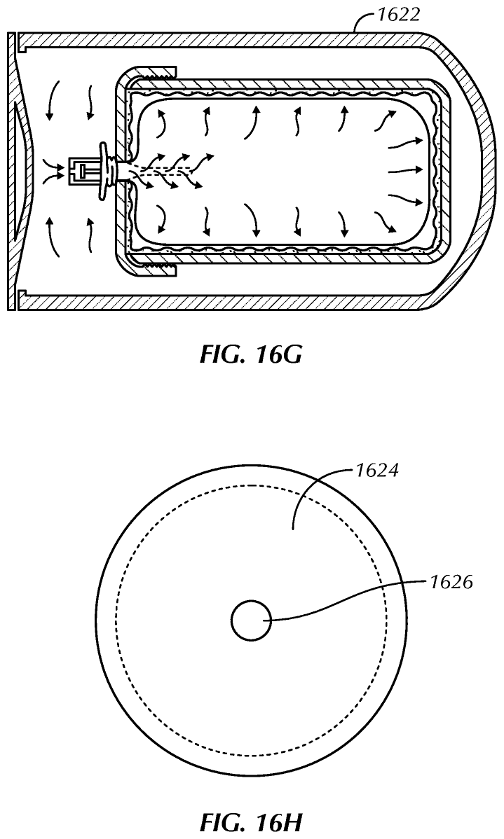

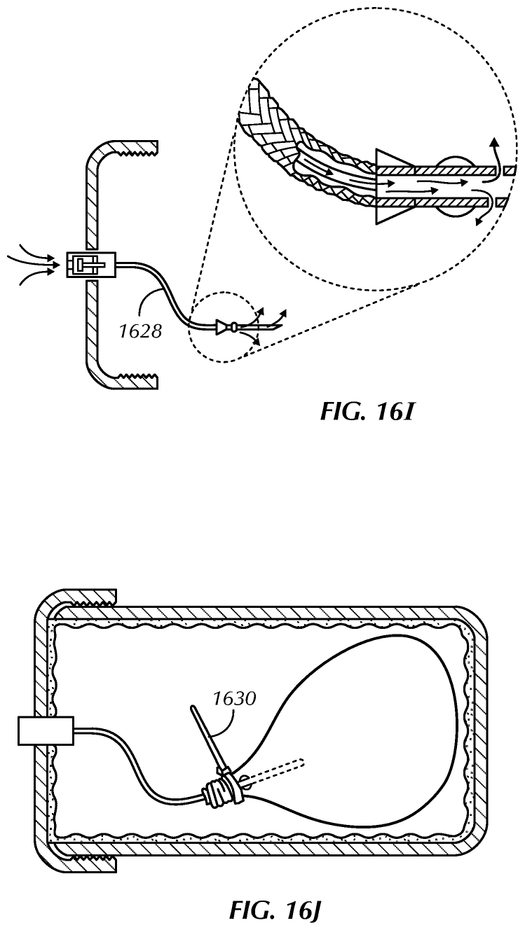

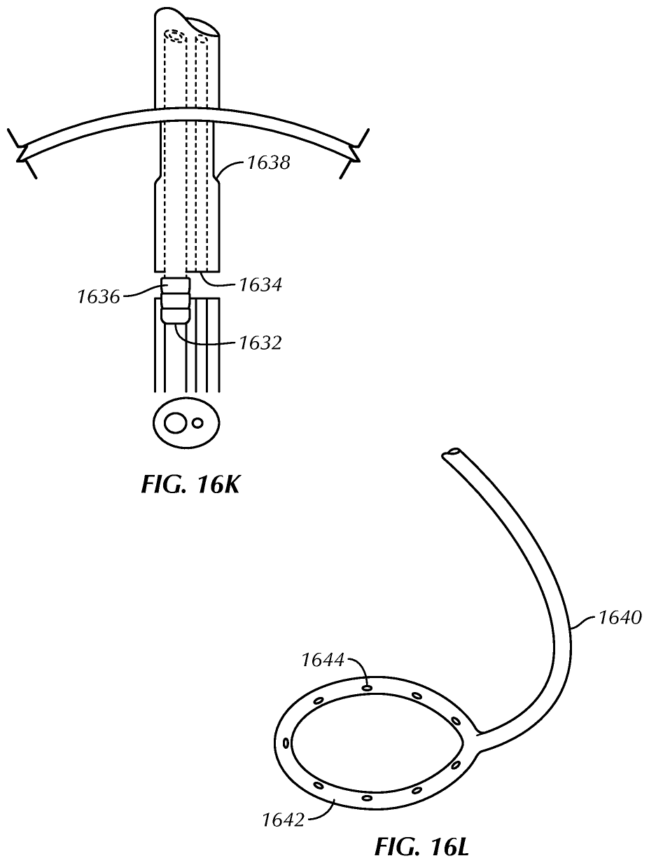

An example sequence for sterilizing infectious medical waste is described below in reference to FIGS. 16A-16J. FIG. 16A shows a metallic container. The metallic container (1600) shown in FIG. 16A is similar to those shown in FIGS. 12A and 12B. However, unlike FIGS. 12A and 12B (which feature a closed metallic cap), FIG. 16A shows the metallic container (1600) having a cap (1602) comprising a through-hole (1604). The metallic container (1600) may be made from rust-proof metallic material like stainless steel, for example. As discussed above, the metallic container (1600) may be coated with protective coating (1606) covering its entire interior. As also discussed above, the thickness, shape, and size of the metallic container (1600) can vary depending on the needs of the individual health institution. The metallic container (1600) is designed such that straight angles in the joints of body of the metallic container (1600) and cap (1602) are prevented. Advantageously, this prevents sharp corners from injuring personnel handling the metallic container (1600).

FIG. 16B shows a top-down view of the through-hole of the cap of the metallic container. The through-hole serves as the only passage between the interior and the exterior of the metallic container.

FIG. 16C shows a compression bag (1608) placed inside a metallic container (1600). The opening (1610) of the compression bag (1608) sits outside of the metallic container (1600), whereas the body of the compression bag (1608) is inside the metallic container. The neck (1612) of the compression bag (1608) is secured and located in the through-hole (1604). At this stage, the neck (1612) of the compression bag (1608) is held tightly enough by the through-hole (1604) such that no medical waste from within the compression bag (1608) can escape the same.

FIG. 16D shows a unidirectional flow device (1614) being introduced into the compression bag (1608) via the bag's opening (1610).

FIG. 16E shows force being applied to disk (1616) of the unidirectional flow (1614) device such that the passage between the interior and the exterior of the metallic container (1600) is open. As a result, water, water vapor, chemical additive, etc., may be introduced into the compression bag (1608) in the first direction (1618) (i.e., into the compression bag (1608)). FIG. 16E shows the compression bag completely inflated due to the fluid pressure that enters the bag through the unidirectional flow device (1616).

FIG. 16F shows the absence of force being applied to the disk of the unidirectional flow device or when the pressure inside the compression bag is greater than that outside the metallic container. As a result, the passage between the interior and the exterior of the metallic container is closed and no infectious medical waste can escape from the compression bag in the second direction (1620) (i.e., out of the compression bag (1608)).

FIG. 16G shows the entire structure of FIG. 16E being placed inside a sterilization autoclave chamber (1622).

FIG. 16H shows the cap (1624) of the metallic container (1600). The through-hole (1626) of the cap (1624) is maintained sealed by the inserted unidirectional flow device (1614).

FIG. 16I shows an alternative form of the unidirectional flow device. Specifically, the unidirectional flow device in FIG. 16I comprises an extension cord (1628). The unidirectional flow device is installed in the through-hole (1626) of the cap (1624). In this embodiment, the unidirectional flow device is firmly secured to the through-hole (1626). For example, this may be accomplished by using a thread mechanism in which the unidirectional flow device is threaded to the through-hole (1626). The extension cord (1628) may be a flexible hose fabricated with stainless steel. The flexible hose is free to move about inside the metallic container (1600) and makes it easier to connect the unidirectional flow device to the compression bag (1608).

FIG. 16J shows the unidirectional flow device working in conjunction with the compression bag (1608). The compression bag (1608) is sealed by a cable tie (1630). The unidirectional flow device is of the type shown in FIG. 16I.

Fluid is (e.g., water, water vapor, chemical additive, etc.) introduced from the sterilization autoclave chamber (1622) into the metallic container (1600) due to pressure differentials. Specifically, pressure outside of the metallic container (1600) is greater than that inside the metallic container (1600).

FIG. 16K shows a connector that may be affixed to the pressure tank. The connector enables injection of water vapor by means of pipe, tube, or flexible hose. Specifically, a pipe, tube or flexible hose may be connected to a channel (1632) and let fluids flow in or out of the pressure tank via the channel (1632). The pipe, tube, or flexible hose may be held to the connector by the protrusions (1636) on the connector. The connector also permits extraction of water vapor or air by means of a passage (1634). The connector may also comprise an edge (1638) that prevents the sealed compression bag from sliding away from the connector.

FIG. 16L shows a tube that can be used to introduce steam from the water vapor generator into the compression bag. The tube comprises a neck (1640) and a loop (1642). The loop (1642) comprises a plurality of pores (1644). The pores (1644) are allocated throughout the loop (1642) so as to evenly distribute the amount of steam in the compression bag and thoroughly sterilize all medical waste therein. Advantageously, the tube enables the steam to reach and sterilize medical waste at various locations throughout the compression bag.

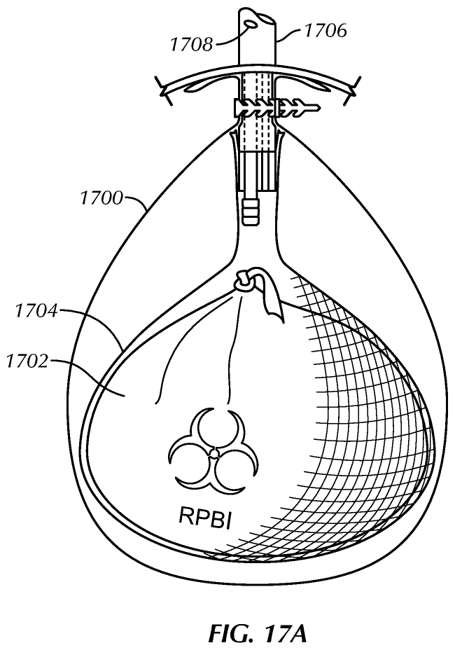

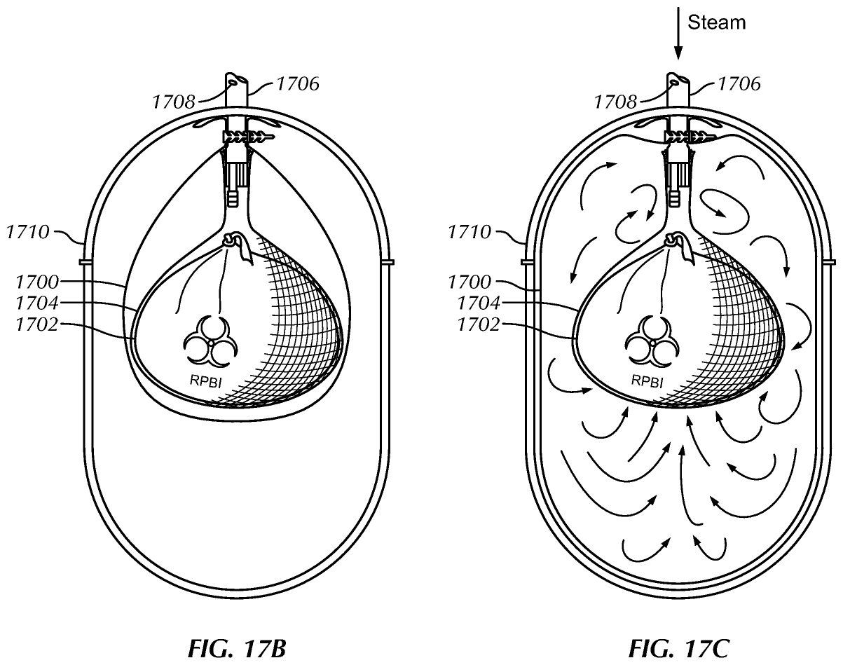

FIG. 17A shows a sealed compression bag holding a hospital red bag that is wrapped in a net, in accordance with one or more embodiments of the present invention. The net (1704) enveloping the hospital red bag (1702) may be gauze, a gauze-like fabric, or any material that allows steam to flow freely, maintains the containment of the hazardous waste within the hospital red bag, and is able to withstand the temperatures of the sterilization system. FIG. 17A also shows an upper portion of the net (1704) and the sealed compression bag (1700) being secured to a connector (1706) having an opening (1708). Attaching the upper portion of the net (1704) to the connector (1706) in this way allows the hospital red bag (1702) containing the hazardous waste to hang inside the compression bag (1700).

Referring now to FIGS. 17A and 17B, in accordance with one or more embodiments of the present invention, the hospital red bag (1702) containing the hazardous waste is wrapped with a net (1704) before inserting the hospital red bag (1702) into the compression bag (1700). Then, the compression bag (1700) containing the hospital red bag (1702) wrapped with the net (1704) is inserted inside the pressure tank (1710), which has the connector (1706) having the opening (1708) affixed thereto. The upper portion of the net (1704) is secured to the connector (1706). The compression bag (1700) is also secured to the connector (1706) and sealed as discussed previously in accordance with one or more embodiments of the present invention. By securing the upper portion of the net (1704) and the compression bag (1700) to the connector (1706) in this way, the hospital red bag (1702) wrapped with the net (1704) hangs inside the compression bag (1700). According to one or more embodiments of the present invention, the hospital red bag (1702) wrapped with the net (1704) remains hanging inside the compression bag (1700) during the sterilization process.

Referring now to FIG. 17C, the structure of FIG. 17B is shown with steam being injected via the connector. Specifically, during the sterilization process, as described herein in accordance with one or more embodiments of the present invention, steam is injected via the opening (1708) of the connector (1706), causing the sealed compression bag (1700) to become inflated. Because the hospital red bag (1702) wrapped with the net (1704) remains hanging inside the compression bag (1700) during sterilization, the entire surface area of the hospital red bag (1702) may be attacked with steam from all directions. Advantageously, wrapping the hospital red bag (1702) with the net (1704) and securing the net (1704) and the compression bag (1700) to the connector (1706) such that the hospital red bag (1702) with the net (1704) hangs inside the compression bag (1700) allows a more homogenous penetration of steam into the hazardous waste contained within the hospital red bag (1702). Moreover, the sterilization temperature of the system is increased, thereby providing increased steam throughout the system.

### Sharps

Sharps (pointed objects) are found in almost every health institution. If treated improperly, they can cause harm to persons and the environment. Existing regulations do not specifically dictate the method for treating sharps. The regulations, however, do require that sharps be made unrecognizable and properly labelled before disposal. The Medical Waste Tracking Act of 1989 further requires that medical waste generators segregate waste at their point of origin and package sharps into rigid, puncture-resistant, leak-resistant containers before transporting off-site.

One or more embodiments of the invention relate to a cartridge for processing sharps (pointed objects) and a method for reducing contaminated sharps into unrecognizable, ordinary waste. The cartridge comprises a protective padding, an inner holder, an outer holder, and a cap.

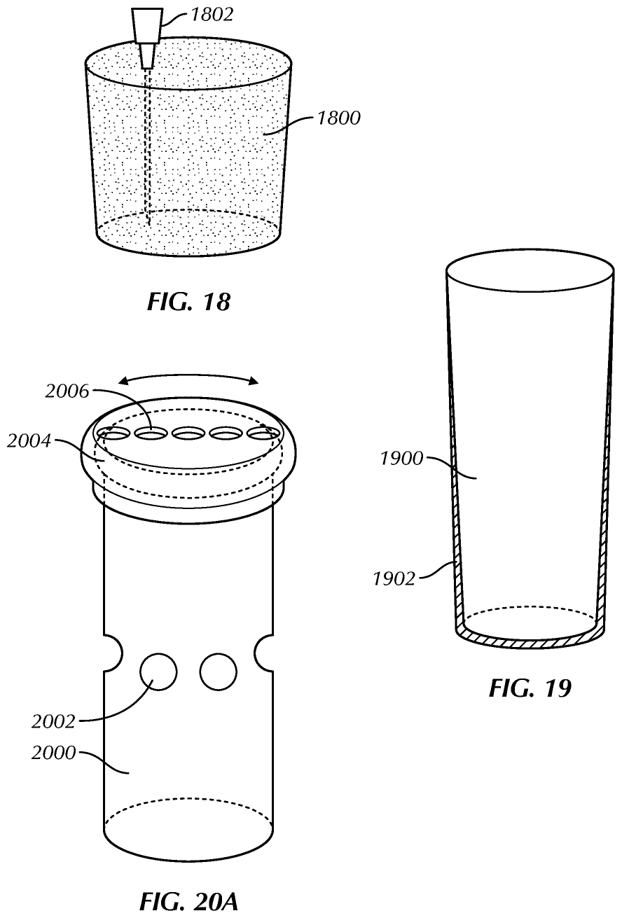

FIG. 18 shows protective padding (1800) that is configured to receive sharps (1802). The protective padding (1800) may be a porous material of foamed-type or expanded-type. Such padding (1800) may be made of foamed or expanded polystyrene, foamed polyurethane or any other material that serves the purposes of receiving and securing sharps (pointed objects). The padding (1800) is to allow the sharps (1802) to perforate easily and remain firmly affixed without moving. Upon engaging the protective padding (1800), the sharps (1802) stand firmly lengthwise. Accordingly, the sharp object (1802) remains immobilized and is oriented correctly during all stages of handling. The protective padding (1800) deforms in high temperature and encapsulates the sharps.

FIG. 19 shows an inner holder (1900) that is a container made from plastic material, such as polyethylene terephthalate (PET), polyvinyl chloride (PVC), polypropylene, polystyrene, polycarbonate, polyethylene, etc. As shown in FIG. 19, the inner holder (1900) is made such that the wall thickness (1902) decreases towards one end. The inner holder (1900) also deforms in high temperature and encapsulates the deformed protective padding.

FIG. 20A shows an outer holder (2000) that is configured to hold the inner holder shown in FIG. 19. The outer holder is a metallic container made of, for example, aluminum, steel, or stainless steel. The metallic container is configured to withstand the high sterilization temperature used to treat sharps and withstand humidity. The outer holder has perforations (2002) that enable thorough treatment of the inner holder shown in FIG. 19. The perforations enable water vapor and warm air to enter the outer holder and treat the inner holder and the inner holder's medical waste content.

FIG. 20A also shows a cap (2004) having through-holes (2006). The cap (2004) may be made of the same or different material as the outer holder (2000). The cap (2004) is to serve as a guide for a user inserting sharps into the outer holder and to prevent objects from escaping from the inner holder. The cap (2004) may be turned clockwise or counterclockwise with respect to the outer holder without being detached from the same. The cap (2004) is attached to the outer holder (2000). The cap (2004) can only be removed from the outer holder (2000) when a user applies a predetermined amount of force. Thus, it is not possible for a child to accidentally detach the cap (2004) from the outer holder (2000). Also, the cap (2004) does not detach from the outer holder due to an inadvertently dropping thereof. The cap (2004) advantageously allows for efficient filling of the cartridge with sharps.

FIG. 20B shows a group of circular through-holes (2006) aligned over a diametrical axis. One of ordinary skill in the art would appreciate that the shape and size of the through-holes can vary depending on the sharps to be inserted.

FIG. 20C shows a cap having non-circular (2006) through-holes. The through-holes are jagged and correspond to a similarly-shaped syringe to be inserted.

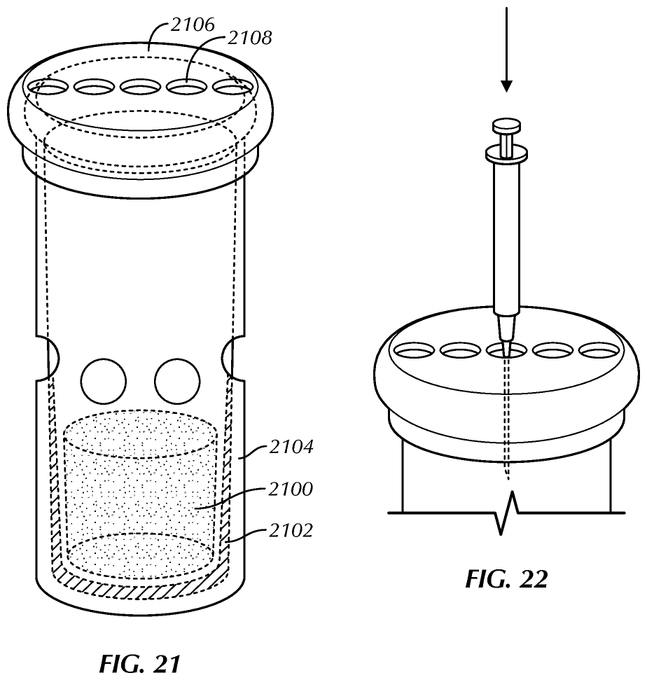

FIG. 21 shows the protective padding (2100) being disposed at a bottom of the inner holder (2102), which is held by an outer holder (2104). The outer holder (2104) comprises a cap (2106) having a plurality of through-holes (2108). The entire structure shown in FIG. 21 may be referred to as a cartridge.

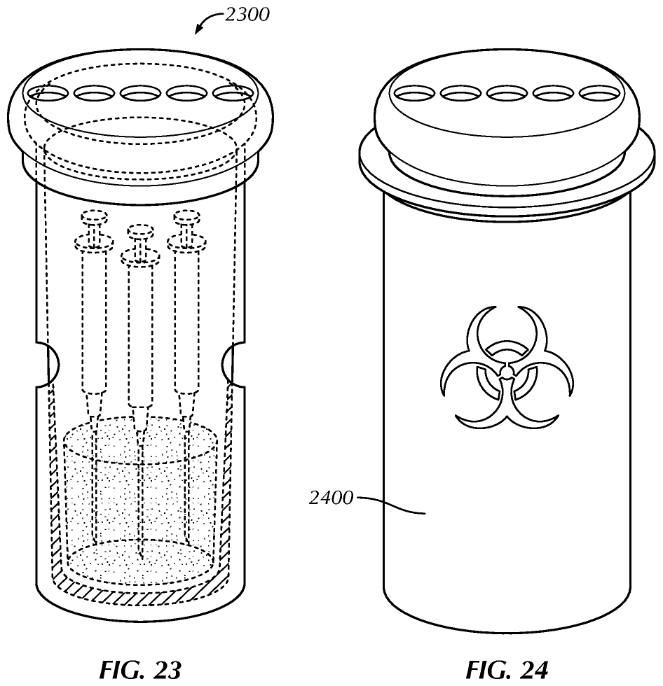

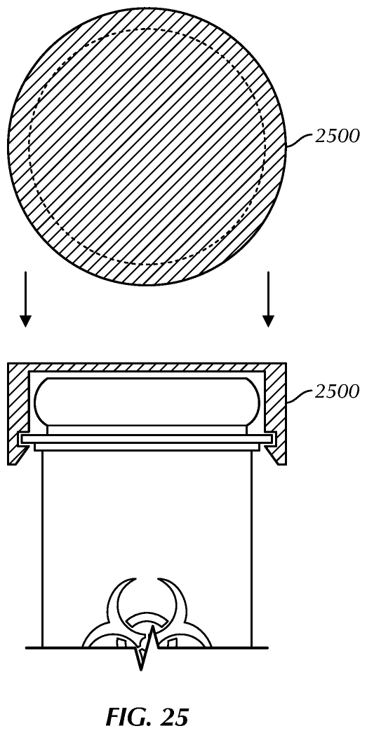

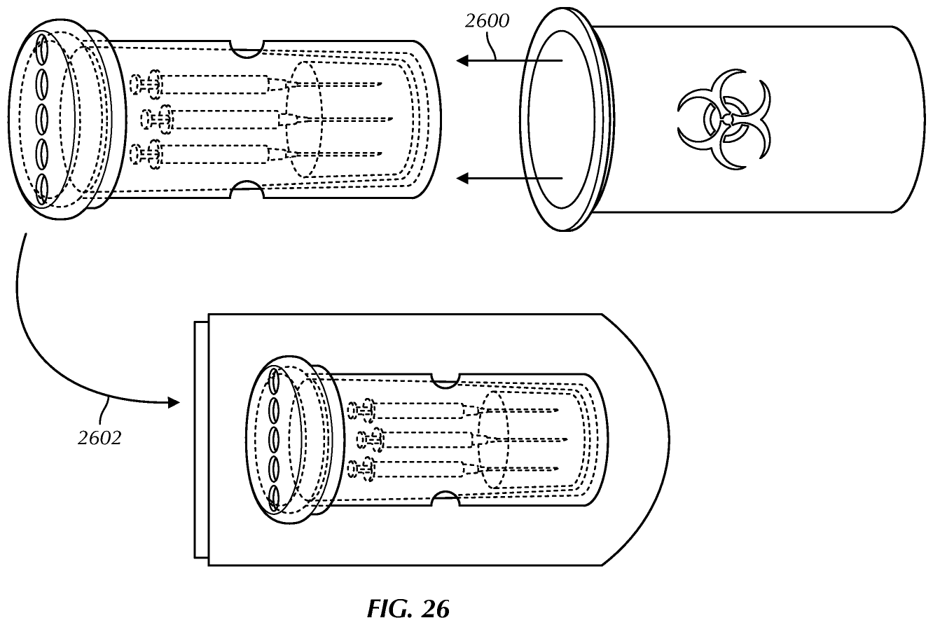

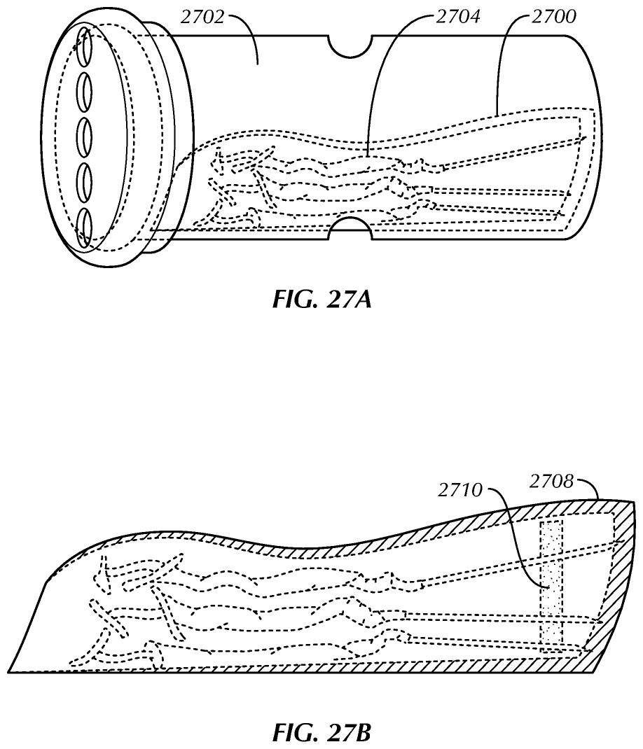

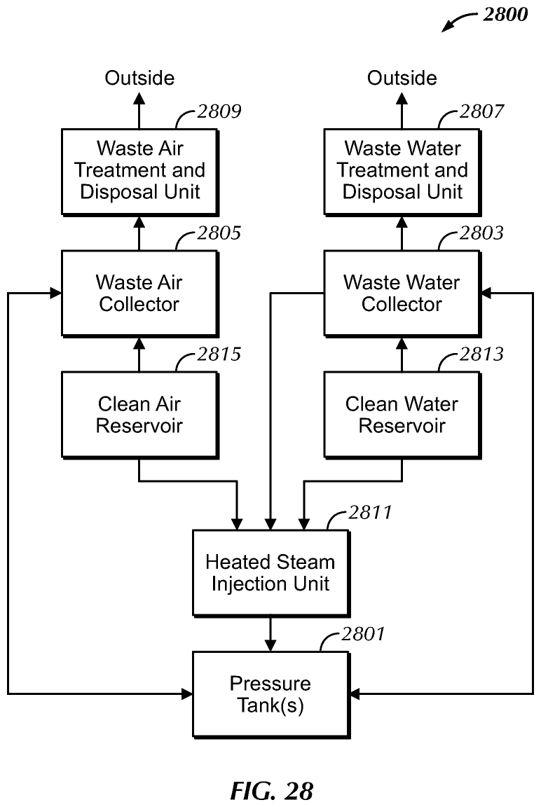

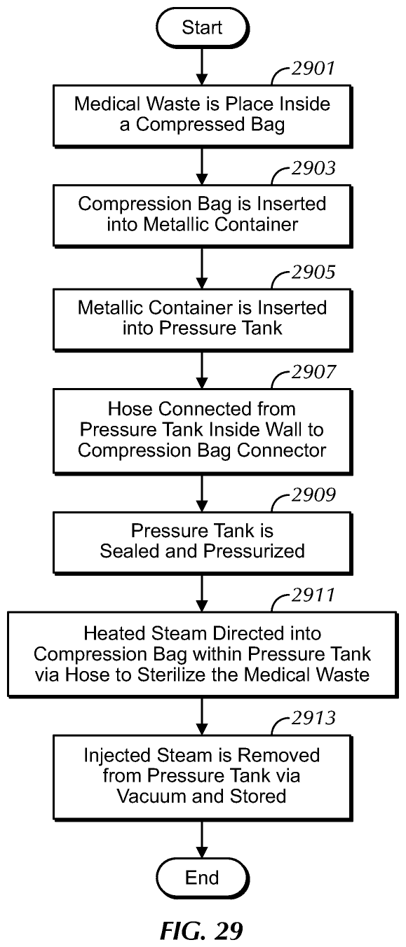

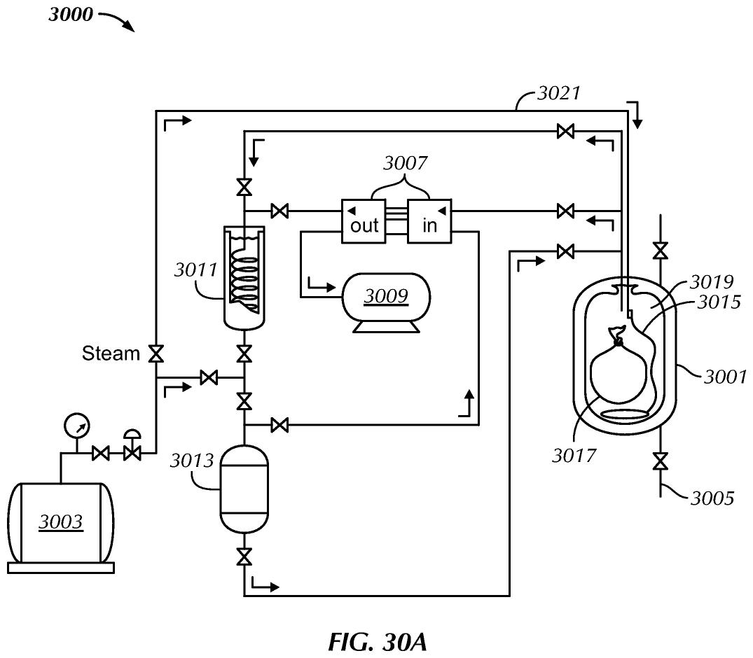

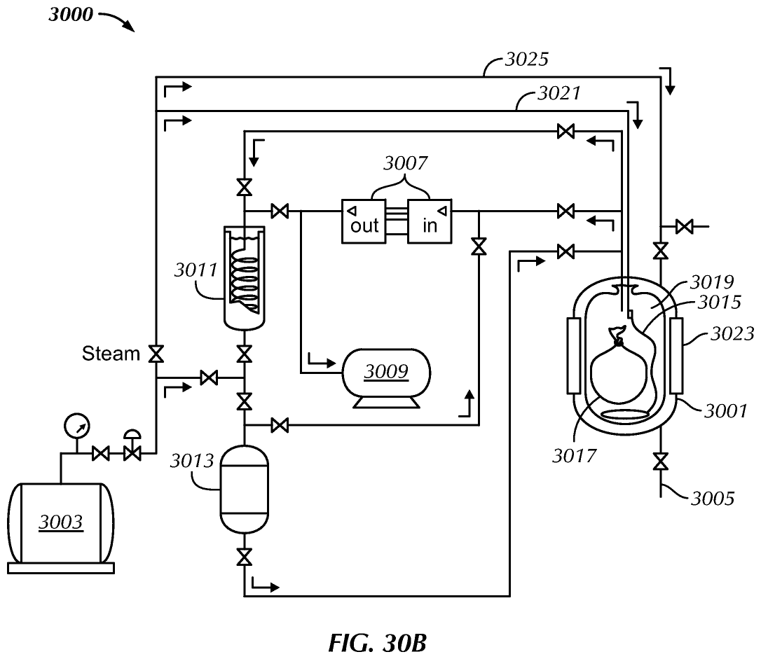

FIG. 22 shows a sharp, pointed object being inserted into a cartridge via one of the cartridge's through-holes.

FIG. 23 shows sharps inserted into a cartridge (300). The sharps are stabilized in their upright position by the protective padding. The cartridge (2300) shown in FIG. 23 is ready to be treated.

FIG. 24 shows a capsule (2400) for holding the cartridge. The capsule (2400) may be labelled with appropriate warning labels. The warning labels may be necessary to comply with government regulations, as set forth above. Those skilled in the art will appreciate that the warning labels may differ based on the government regulations in place where the capsule is being used. For example, in one or more embodiments, the capsule (2400) may have a minimum resistance of 12.5 N in all of its components (determined by measuring the strength required to perforate sides and bottom with a 21×32 mm-gauge hypodermic needle by means of strength gauge or tensometer).

FIG. 25 shows an encapsulation mechanism that complies with the requirements of “Safe-Ensemble Cap with Permanent Closing” for all containers holding sharps. FIG. 25 shows a red cap (2500) (made of polypropylene, for example) offering a safe ensemble with a permanent closing mechanism. The cap is inserted over the capsule (2400) shown in FIG. 24 and attaches with a closing mechanism so as to contain the cartridge (2300) fully. In one or more embodiments, the closing mechanism may be a coupling lock, as shown in FIG. 25. Alternatively, in one or more embodiments, the closing mechanism may be a threaded cap. The closing mechanism is not limited, so long as the red cap (2500) is able to be fixed to the capsule shown in FIG. 24 and allows for prevention of accidental opening of the capsule. In one or more embodiments, to allow insertion of additional sharps into a capped capsule (2400), the red cap (2500) may be removable so that the additional sharps may be inserted via the through-holes of the cartridge (2300) contained within.

FIG. 26 shows the process of removing (2600) a cartridge from a capsule and placing (2602) the cartridge into a sterilization autoclave chamber. The cartridge may be directly placed into the sterilization autoclave chamber or may be placed into the compression bag described above. The compression bag may or may not be placed into a metallic container, as also described above. Once the cartridge (2300) is emptied from the capsule (2400), a new cartridge may be inserted into the capsule. Accordingly, in one or more embodiments, the capsule may be reused.

In one or more embodiments, once placed inside a sterilization autoclave chamber, the cartridge is subjected to a sterilization cycle by saturated vapor in a temperature range between 100 and 150 degrees Celsius. According to one or more embodiments of the present invention, the corresponding pressure for the aforementioned temperature range is between 100 and 500 KPa. In other embodiments, the corresponding pressure for the aforementioned temperature range is between 101 KPa and 476 KPa.

In one or more embodiments, the sterilization autoclave is equipped with a heating system (e.g., a radiation/thermal conduction unit, a warm air convection unit, etc.). The heating system is capable of heating the interior of the autoclave to between 100 and 200 degrees Celsius. Sharps subjected to this temperature range deform. In particular, the temperature range exceeds the thaw point of various plastic materials that make up the various sharps. Thus, the proposed treatment method complies with the regulation requiring that sharps be made unrecognizable after treatment. For the purpose of this application, the term “unrecognizable” is defined as “the loss of physical and biological-infectious characteristics of an object to not be reused.”

In one or more embodiments, once placed inside a sterilization autoclave chamber, the cartridge (2300) is first subjected to a sterilization cycle by saturated vapor in a temperature range between 150 and 200 degrees Celsius. Subsequently, the cartridge (2300) is subjected to a dry heating cycle in a temperature range between 100 and 200 degrees Celsius. The treatment time for each portion or the combination (vapor treatment and dry heating) may be between 1 and 120 minutes. As with treating infectious medical waste, the sterilization autoclave can be customized with various settings, including temperature, amount of time for sterilization, etc. Vapor treatment sterilizes the sharps; dry heating deforms the sharps and makes them unrecognizable. After treatment, the deformed cartridge (2300) may be cooled to between 45 and 50 degrees Celsius to enable safe removable from the sterilization autoclave chamber.

When the vapor sterilization process is complete, the sterilization autoclave chamber's door may automatically open slightly to allow the remaining water to escape and prevent pressure from developing inside before the dry heating process begins. The sterilization autoclave chamber may be any shape and size. The sterilization autoclave chamber may have a capacity that is at least 5 liters. According to one or more embodiments of the present invention, the sterilization autoclave chamber may have a capacity that is between 20 and 40 liters.

In one or more embodiments of the invention, once placed inside a sterilization autoclave chamber, the cartridge (2300) is only subjected to a dry heating cycle in a temperature between 100 and 200 degrees Celsius. Different from the aforementioned procedures, the vapor treatment may not be used. Accordingly, one of ordinary skill in the art would appreciate that, in one or more embodiments, vapor treatment, chemical treatment, and dry heating may be used in various combinations to sterilize medical waste without departing from the spirit of the invention.

FIG. 27A shows a deformed inner holder (2700) after having been subjected to treatment. The integrity of the outer holder (2702) is still in place as it is made of metallic components. The sharps (2704) inside the inner holder, however, are deformed and made unrecognizable.

FIG. 27B shows a detailed version of FIG. 27A. Notably, the sharp ends of the pointed object (2704) are embedded in the inner holder's thick side (2708), but do not perforate the same. FIG. 27B also shows the deformed sharps being enveloped by the deformed protective padding (2710). Because the deformed sharps (2704) are unrecognizable and, importantly, are impossible to use for their intended purposes, embodiments of the invention comply with regulations requiring “loss of physical and biological-infectious characteristics of the object to not be used again.”

FIG. 28 shows a block diagram of a sterilization system (2800) in accordance with one or more embodiments of the present invention. The sterilization system (2800) includes one or more pressure tanks (2801) for containing medical waste to be sterilized. The pressure tank(s) (2801) are connected to containment units for waste water and water air, i.e., waste water collector (2803) and waste air collector (2805). Each of waste water collector (2803) and waste air collector (2805) may be respectively connected to a waste water treatment and disposal unit (2807) and a waste air treatment and disposal unit (2809). Those skilled in the art will appreciate that, depending on a particular jurisdiction in which the sterilization system (2800) is being operated, there may be different requirements for the treatment of waste air and waste water prior to disposal or release from the closed sterilization system (2800). For example, waste air or water may be burned, injected with chemicals, or otherwise rendered inert prior to release, and/or may be required to be released under specific conditions.

The sterilization system (2800) also includes a unit for supplying heated steam to the pressure tank(s) (2801), i.e., heated steam injection unit (2811). As described above, the heated steam is injected into the pressure tank(s) (2801) in one or more cycles so as to sterilize medical waste contained within the pressure tank(s) (2801). After each cycle, the water and air contained within the pressure tank(s) (2801) is collected in the waste water collector (2803) and waste air collector (2805). Those skilled in the art will appreciate that some water and air will be lost or gained at each opening and replacement of the medical waste in the pressure tanks. Accordingly, each of the waste water collector (2803) and waste air collector (2805) respectively contain connections to a clean water reservoir (2813) and clean air reservoir (2815).

The clean water reservoir (2813) and the clean air reservoir (2815) add clean water and fresh air, respectively, to the sterilization system (2800) to enable satisfactory sterilization of the medical waste. The frequency for the adding varies and may be, for example, once every cycle or several times per cycle. Additionally, the clean water reservoir (2813) and the clean air reservoir (2815) may be directly connected to the heated steam injection unit (2811), as it may be desirable in one or more embodiments to perform the sterilization process in at least one cycle with clean air and/or water.

The various reservoirs and components in the block diagram may be connected via a pipe system, tubes, or any other transportation means. As shown by the arrows in the block diagram, certain connections within the system may be bidirectional while others are unidirectional. As discussed above, those skilled in the art will appreciate various modifications, for example, in the flow of the air and water throughout the sterilization system (2800) without departing from the spirit of the invention. For example, the bidirectional connections between the pressure tank(s) (2801) and, respectively, the waste water collector (2803) and waste air collector (2805) could be made unidirectional away from the pressure tank(s) (2801). By doing so, all connections within the system would only allow flow in a single direction. In any case, the sterilization system (2800) is constructed such that the air and water employed in the sterilization process is contained within a closed loop until properly treated for release from the sterilization system (2800). By controlling the process in this fully closed manner, the improper release of any contaminants of the medical waste from the sterilization system (2800) can be surely avoided.

FIG. 29 is a flow chart detailing a method of sterilizing medical waste in accordance with one or more embodiments of the invention. Medical waste is placed inside a compression bag (step 2901). Then, the compression bag is inserted into a metallic container (step 2903). The metallic container is inserted into pressure tank (step 2905). A hose is connected from an inside wall of the pressure tank to the compression bag connector (step 2907). Then, the pressure tank is sealed and pressurized (step 2909). Once water vapor has been generated and heated to steam, the heated steam is directed into the compression bag within pressure tank via the hose to sterilize the medical waste contained inside (step 2911). Finally, the injected steam is removed from pressure tank via vacuum and stored (step 2913). Those skilled in the art will appreciate that the above steps may be repeated for several cycles. Furthermore, although each step of the example sequence is described sequentially, one of ordinary skill in the art would appreciate that the steps may be executed in parallel, that some steps may be omitted, and that the ordering of the steps may be rearranged depending on the nature of the medical waste. Each step will be discussed in detail below with reference to the components of the sterilization system shown in FIG. 30A.

FIG. 30A shows a sterilization system (3000), in accordance with one or more embodiments of the invention, for sterilizing medical waste. The sterilization system (3000) comprises a pressure tank (3001), a water vapor generator (3003), valves (3005), a vacuum compressor (3007), an accumulation tank (3009), a condenser (3011), a recipient (3013), a flexible hose (3015), and pipes (3021).

The pressure tank (3001) may be of any shape and size. The pressure tank (3001) may be made from stainless steel, elemental metal, or any alloys. The pressure tank (3001) is able to withstand operating temperatures of around 250 degrees Celsius. Regular operating temperatures of the pressure tank range between 120 and 200 degrees Celsius. The pressure tank (3001) is configured to receive a metallic container. Medical waste (3017) is placed inside a compression bag (3019), which is placed inside the metallic container.

The water vapor generator (3003) may comprise a heater that heats water to around 200 degrees Celsius, thereby evaporating water to form steam. The water vapor generator (3003) directs the steam into the compression bag (3019) by means of a pipe, tube, flexible hose (3015), etc. Once inside the compression bag (3019), a microclimate is created to allow sterilization of the medical waste (3017). The water vapor from the water vapor generator (3003) exerts a pressure inside the compression bag that ranges between 1 and 5 kg cm⁻². As discussed above, the operating temperature ranges between 120 and 200 degrees Celsius. More specifically, the operating temperature ranges between 121 and 150 degrees Celsius. The amount of time needed to sterilize the medical waste (3017) can be adjusted depending on the type of medical waste.

The pipe, tube, flexible hose (3015), etc., may be unidirectional or may be bidirectional. In the event that the pipe or the flexible hose is bidirectional, waste water (i.e., water condensed from vapor that was used to treat medical waste) may exit via the same pipe, tube, or flexible hose (3015) and be stored in the recipient (3013). The vapor may be condensed by the condenser (3011), which could be a heat exchanger. Alternatively, the vapor may be condensed by coolants, including Freon. The pipe, tube, or flexible hose (3015) is introduced inside the compression bag via an opening of the same and the pipe, tube, or flexible hose (3015) may be in contact with the medical waste. The material of the pipe, tube, or flexible hose (3015) is not limited so long as the pipe and the flexible hose are able to retain their physical and operating characteristics during the operating temperatures of the pressure tank (3001).

The vacuum compressor (3007) vacuums contaminated air from the pressure tank (3001) and stores the same in the accumulation tank (3009). To enable multiple sterilization cycles, the volume of the contaminated air is compressed to save space in the accumulation tank (3009). The valves (3005) in the system govern the flow of fluids, including air.

In one or more embodiments, the sterilization system (3000) may be sized appropriately to fit all of the components on a wheeled trolley, cart, or the like capable of being moved throughout a hospital. In one or more embodiments, the sterilization system (3000) may be sized in larger so as to be capable of holding several compression bags at a single time.

In one or more embodiments, the sterilization system (3000) may be located in a single location containing each of the components of the system, or various components may be located at different locations and are merely in connection with one another to perform the required functions. In one or more embodiments, certain components of several such sterilization systems (3000) may be distributed and connected to a single component. For example, several pressure tanks may be located in different locations, such as within individual hospital rooms or in a particular room on individual hospital floors, and the several pressure tanks are each in connection with a single water vapor generator, vacuum compressor, accumulation tank, condenser, recipient in a different location, such a basement of the hospital, roof of the hospital, or other nearby location. Those skilled in the art will appreciate that combinations of the above-described sterilization systems (3000) may also be possible. For example, one or more of the components of the system may be sized so as to fit on a wheeled trolley, cart, or the like, capable of connecting to the remaining components via connections installed in one or more rooms of a hospital.

Now, the method described in FIG. 29 will be described in terms of specific exemplary steps with reference to the system described in FIG. 30.

Step 1. Once the pressure tank (3001) has been loaded and sealed, water vapor is injected from the water vapor generator (3003) into the recipient (3013). This way, the water vapor pushes the condensates from the previous cycle inside the recipient towards the interior of the compression bag (3019). Depending on whether the pipe, tube, or flexible (3015) is used, the water vapor is sent to the bottom of the compression bag (3019) or into specific portions of the compression bag (3019). In any event, the medical waste (3017) is broken down and sterilized inside the compression bag (3019). In one or more embodiments, the approximate duration of this step may be between 1 to 4 minutes and the pressure of the compression bag may be approximately 3 kg cm⁻².

Step 2. Next, one or several cycles of vapor extraction and vacuum generation steps are performed so as to guarantee total extraction of air from the sterilization system (3000). Each cycle starts with a vapor extraction step that extracts liquid contained inside the compression bag (3019). The vapor is extracted using the pipes (3021) in the system and transported to the condenser (3011) (and into the recipient (3013)). In one or more embodiments, the extraction operation continues until the pressure inside the compression bag is reduced to 0.5 kg cm⁻². The extraction process may be carried out using the vacuum compressor (3007). With respect to the vacuum generation step, the pipes are used to vacuum the compression bag such that condensates are also removed from the compression bag. Once removed, the condensates flow towards the recipient (3013). In one or more embodiments, the vacuum process continues until a value of, for example, −80 Kpa is reached inside the compression bag.

Step 3. Once the first extraction/vacuum cycle is completed, steam is injected inside the compression bag. In this step, the water vapor is injected into the compression bag by means of a pipe, tube, or flexible hose (3015). In one or more embodiments, the pressure of the supplied vapor in this process may be, for example, 3.5 kg cm⁻² and the exposure time may be, for example, 5 minutes.

Step 4. After injection of steam, another extraction/vacuum cycle is performed. And, after the new extraction/vacuum cycle, additional steam is injected as in Step 3.

Step 5. After repeating Steps 2-4, the sterilization system (3000) performs one more extraction/vacuum cycle (which is followed by another steam injection cycle). However, in Step 5, the steam injection temperature reaches 134 degrees Celsius and the exposure time is around 15 minutes. As discussed above, various parameters, including time, temperature, volume, pressure, etc., may all vary depending on the nature of the medical waste (3017).

Step 6. The sterilization process is now complete and the system is ready to depressurize the compression bag, thereby removing the remaining vapor trapped inside the compression bag. As with other condensates, the remaining vapor is condensed and directed to the recipient (3013). The condensates inside the recipient (3013) can be used to sterilize a new batch of medical waste (2917) in another cycle.

Step 7. When the pressure of the system has been discharged, one last vacuum process may be performed to compress the sterile materials inside the compression bag (3019).

Step 8. In one or more embodiments, when the temperature of the system lowers to 85 degrees Celsius, the pressure tank is opened. Then, in one or more embodiments, when the temperature of the system lowers to 70 degrees Celsius, the compression bag (3019) may be removed for disposal. In one or more embodiments, the sterilization system (3000) may include indicators to alert users of the stages of operation and/or properties, e.g., temperature, pressure, humidity, etc., within the pressure tank (3001). Thereafter, the system is available for use by a new batch of medical waste.

FIG. 30B shows a sterilization system (3000) for sterilizing medical waste according to one or more embodiments of the present invention. Similar to the sterilization system shown in FIG. 30A, the sterilization system (3000) shown in FIG. 30B also comprises a pressure tank (3001), a water vapor generator (3003), valves (3005), a vacuum compressor (3007), an accumulation tank (3009), a condenser (3011), a recipient (3013), a flexible hose (3015), and pipes (3021), the description of which will not be repeated here.

In addition to these, the sterilization system (3000) of FIG. 30B includes one or more heating resistances (3023) attached to the walls of the pressure tank (3001). The heating resistances (3023) may be any type of heating system that is capable of providing the pressure tank (3001) with an effective and favorable environment for sterilization as understood by those skilled in the art. Before injecting steam into the pressure tank (3001) for the sterilization process, the one or more heating resistances (3023) may be heated to a desired temperature to prepare the pressure tank (3001) for hot sterilization, thereby increasing the sterilizing effectiveness of the steam. According to one or more embodiments of the present invention, the one or more heating resistances (3023) have a temperature range from 0 to 380 degrees Celsius. In one or more embodiments, the one or more heating resistances (3023) may be heated to 80 degrees Celsius. Once turned on, the one or more heating resistances (3023) may remain on for the remainder of the sterilization process.

As also shown in FIG. 30B, the sterilization system (3000) may include an additional conduit or pipe (3025) for steam installed on a top part of the pressure tank (3001). According to one or more embodiments of the present invention, the additional conduit (3025) provides for the injection of steam inside the pressure tank (3001), but not inside the compression bag (3019). During the sterilization process, steam may be injected into the pressure tank (3001) via the conduit (3025) to increase the temperature of the environment inside the pressure tank (3001) and outside the compression bag (3019). According to one or more embodiments of the present invention, steam may be injected into the pressure tank (3001) via the conduit (3025) for 3 to 5 minutes before any steam is introduced into the compression bag (3019). Heating up the pressure tank (3001) in this way creates an environment where the steam may more effectively sterilize the medical waste (3017) during the sterilization process. After injecting steam into the pressure tank (3001) via the conduit (3025) for 3 to 5 minutes, steam may be cleared out of the pressure tank (3001) and into the condensation system, which includes the condenser (3011) and the recipient (3013), as previously described with respect to FIGS. 29 and 30A. Thereafter, steam may be injected inside the compression bag (3019) by means of a pipe, tube, or flexible hose (3015), thereby inflating the compression bag (3019). In one or more embodiments, the pressure of the supplied vapor in this process may be, for example, 3.3 kg/cm², and the exposure time may be, for example, 12 minutes. After injection of steam inside the compression bag (3019), steam may be cleared out into the condensation system for about 30 seconds, for example. This cycle of injecting/clearing out steam may be repeated as needed in accordance with one or more embodiments of the present invention. After additional steam is injected into the compression bag (3019) for the last time of the sterilization process, the steam is released into the condensation system. According to one or more embodiments of the present invention, heating resistances (3023) that were turned on during sterilization may now be turned off, and the pressure tank (3001) may be opened when the temperature of the system lowers to 85 degrees Celsius, for example. After the sterilization system (3000) has sufficiently cooled down, to for example 70 degrees Celsius, the compression bag (3019) may be removed for disposal.

According to one or more embodiments of the present invention, the sterilization system (3000) may include the one or more heating resistances (3023) and the additional conduit (3025) for steam, either alone, in combination, or not at all, without departing from the scope of the present disclosure.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A system for sterilizing medical waste comprising:
a pressure tank configured to receive a compression bag having the medical waste;
a water vapor generator that introduces steam into the pressure tank via a first pipe using a connector attached to the pressure tank; and
a vacuum compressor that removes fluids from the pressure tank,
wherein the pressure tank, water vapor generator, and vacuum compressor are connected in a closed manner such that the fluids within the pressure tank are contained.

2. The system according to claim 1 further comprising a condenser that condenses vapor used to sterilize the medical waste.

3. The system according to claim 1, further comprising an accumulation tank, wherein the vacuum compressor compresses contaminated air from within the pressure tank for storage within the accumulation tank.

4. The system according to claim 1, wherein the steam is introduced into the compression bag via the first pipe using a flexible hose connected to the connector.

5. The system according to claim 1, wherein the connector is bidirectional.

6. The system according to claim 1 further comprising a recipient that is configured to store condensates used to sterilize the medical waste for re-use in a subsequent sterilization cycle.

7. The system according to claim 6, wherein pipes are used to transport the condensates to the recipient.

8. The system according to claim 1, wherein the compression bag is sealed using a cable tie having a plurality locking positions.

9. The system according to claim 8, wherein a first distance between a first locking position and a second locking position is different from a second distance between the second locking position and a third locking position.

10. The system according to claim 1 further comprising at least one heating resistance attached to a wall of the pressure tank.

11. The system according to claim 1, wherein the compression bag having the medical waste comprises the medical waste contained within a bag that is wrapped with a net.

12. The system according to claim 1, further comprising a second pipe installed on a top part of the pressure tank, wherein the water vapor generator further introduces steam into the pressure tank via the second pipe, but does not introduce steam into the compression bag via the second pipe.

13. The system according to claim 12, wherein the water vapor generator introduces the steam into the compression bag via the first pipe using a flexible hose connected to the connector.

14. A method for sterilizing medical waste comprising:
placing a compression bag having medical waste inside a pressure tank;
connecting the compression bag to a connector within the pressure tank;
extracting fluids from the compression bag to create a vacuum-like environment therein;
introducing steam into the compression bag;
removing the steam from the pressure tank;
storing the fluids extracted from the compression bag and the steam removed from the pressure tank; and
cooling the compression bag within the pressure tank.

15. The method according to claim 14, wherein the vacuum-like environment comprises pressure of −80 Kpa or lower.

16. The method according to claim 14, wherein the introducing comprises introducing the steam at a pressure of 3.5 kg cm⁻².

17. The method according to claim 14, wherein the introducing steam and the removing of the steam are repeated for a predetermined number of cycles before cooling the compression bag.

18. The method according to claim 17, wherein the steam removed from the pressure tank is re-used in a subsequent cycle.

19. The method according to claim 14, wherein the compression bag having the medical waste comprises the medical waste contained within a bag that is wrapped with a net.

20. The method according to claim 19, further comprising connecting an upper portion of the net to the connector within the pressure tank such that the wrapped bag containing the medical waste hangs inside the compression bag.

21. The method according to claim 14, further comprising introducing steam into the pressure tank and not into the compression bag before the step of introducing steam into the compression bag.

22. The method according to claim 14, further comprising turning on at least one heating resistance attached to a wall of the pressure tank before at least the step of introducing steam into the compression bag; and turning off the at least one heating resistance before the step of cooling the compression bag within the pressure tank.

23. A sterilization system comprising:
a pressure tank for containing medical waste to be sterilized;
a waste water collector and waste air collector connected to the pressure tank; and
a heated steam injection unit that injects heated steam into the pressure tank in one or more cycles so as to sterilize medical waste contained within the pressure tank,
wherein, after each cycle, the water and air contained within the pressure tank is collected in the waste water collector and the waste air collector.

24. The sterilization system of claim 23 further comprising a clean water reservoir connected to the waste water collector; and a clean air reservoir connected to the waste air collector.

25. The sterilization system of claim 24, wherein the clean water reservoir and the clean air reservoir are also connected to the heated steam injection unit.

26. The sterilization system of claim 23 further comprising a waste water treatment and disposal unit connected to the waste water collector; and waste treatment and disposal unit connected to the waste air collector.

27. The sterilization system of claim 23, wherein the connections among the waste water collector, the waste air collector, the pressure tank, and a heated steam injection unit are unidirectional.

28. The sterilization system of claim 23, wherein the water and air collected within the waste water collector and the waste air collector is re-used in a subsequent cycle.
